(19) United States
(12) Patent Application Publication
(10) Pub. No.: US 2026/0354974 A1
(43) Pub. Date: Nov. 12, 2026

(54) DEVICE FOR TREATING VALVULAR INSUFFICIENCY AND METHOD OF DELIVERING SAME

(71) Applicant: CORAMAZE TECHNOLOGIES LTD., Petach Tikva (IL)

(72) Inventors: Boaz HARARI, Ganei Tikva (IL); Ido SADAN, Tel-Aviv (IL); Merav TAMIRI, Tel Aviv-Jaffa (IL); Vadim GORDON, Petah Tikva (IL); Nir KAFRI, Rosh HaAyin (IL); Ariella NAUMBURGER, Tel Aviv (IL)

(73) Assignee: CORAMAZE TECHNOLOGIES LTD., Petach Tikva (IL)

(21) Appl. No.: 19/135,321

(22) PCT Filed: Dec. 4, 2023
(86) PCT No.: PCT/IL2023/051241

§ 371 Date: Jun. 3, 2025

Related U.S. Application Data
(60) Provisional application No. 63/430,085, filed on Dec. 5, 2022.

(51) Int. Cl. A61F 2/24

(52) U.S. Cl. CPC A61F 2/246; A61F 2/2466; A61F 2220/0008; A61F 2230/0004

(57) ABSTRACT

A device for treating valvular insufficiency that includes an expandable anchor attached to a transvalvular spacer and a delivery system for delivering the device and enabling deployment and positioning of the transvalvular spacer prior to anchoring.

### RELATED APPLICATION/S

This application claims the benefit of priority of U.S. Provisional Patent Application No. 63/430,085, filed on Dec. 5, 2022, the contents of which are incorporated herein by reference in their entirety.

### BACKGROUND

The present invention relates to a device for treating valvular insufficiency and to catheter systems for delivering and retrieving the device. Embodiments of the present invention relate to a device that includes a trans-valvular spacer attached to a non-traumatic anchor positionable within the atrium and to a delivery system that enables deployment and positioning of the spacer prior to device anchoring.

Valvular insufficiency is a cardiac disease characterized by the failure of a cardiac valve to fully close leading to valvular regurgitation or leakage.

Anatomically, the valves are part of the dense connective tissue of the heart known as the cardiac skeleton and are responsible for the regulation of blood flow through the heart and great vessels. Valvular insufficiency due to failure or dysfunction can result in diminished heart functionality and a decrease in blood flow through the body. Treatment of damaged valves may involve medication alone, surgical valve repair (valvuloplasty) or replacement (insertion of an artificial heart valve).

Atrioventricular valvular insufficiency can lead to blood leakage or flow from the ventricle back into the atrium (regurgitation), rather than being forced out of the ventricle upon contraction. Valve regurgitation is a common valvular insufficiency and is typically treated via valve replacement or valve repair through open heart surgery or a minimally invasive (percutaneous) procedure.

While percutaneous valve replacement/repair is less traumatic to the patient and can be used in patient populations that are not candidates for open heart surgery (due to age or co-morbidities), there are still challenges with delivering, and positioning the implant and with the stability and function of the implant in the heart over extended time periods.

There is thus a need for, and it would be highly advantageous to have, a device for correcting valvular insufficiency and a system for delivering and retrieving the device.

### SUMMARY OF THE INVENTION

According to one aspect of the present invention there is provided a device for treating valvular insufficiency comprising a transvalvular spacer for providing a coaptation surface to leaflets of a heart valve; and an expandable anchor attached to the transvalvular spacer and being for positioning the transvalvular spacer across the heart valve when expanded in a heart chamber, the expandable anchor having a proximal connector attachable to a first element of a delivery system and a distal connector attachable to a second element of a delivery system, the first and second elements being moveable in opposite directions to expand the expandable anchor.

According to embodiments of the present invention the transvalvular spacer maintains a position relative to the heart valve when the first element and the second element move in the opposite directions.

According to embodiments of the present invention a degree of movement of the first element and the second element determines an extent of expansion of the anchor.

According to embodiments of the present invention the device further comprises a coupling element for engaging a retrieval catheter.

According to embodiments of the present invention the coupling element is configured for engagement via a lasso or snare.

According to another aspect of the present invention there is provided device for treating valvular insufficiency comprising: a transvalvular spacer for providing a coaptation surface to leaflets of a heart valve; and an expandable anchor attached to the transvalvular spacer and being for positioning the transvalvular spacer across the heart valve when expanded in a heart chamber, the expandable anchor including a plurality of struts, wherein some of the struts include a flexible portion for enabling the transvalvular spacer to tilt with respect to the expandable anchor when expanded.

According to embodiments of the present invention the flexible portion is an undulating portion. This flexible joint between anchor and spacer allows the atrial portion to maintain static posture while the spacer is free to fill the coaptation gap between the native leaflets during ventricular systole.

According to another aspect of the present invention there is provided a method of treating valvular insufficiency comprising: deploying within a heart valve a transvalvular spacer having a balloon for providing a coaptation surface to leaflets of the heart valve; inflating the balloon within the heart valve to a volume that meets a coaptation goal; and expanding an expandable anchor attached to the transvalvular spacer within a heart chamber to thereby anchor the transvalvular spacer across the heart valve.

According to embodiments of the present invention the transvalvular spacer maintains a position relative to the heart valve when the expandable anchor is expanded.

According to embodiments of the present invention the method further comprises deflating and optionally reinflating the balloon.

According to embodiments of the present invention the expandable anchor further comprises a coupling element for engaging a retrieval catheter.

According to embodiments of the present invention the method further comprises deflating the transvalvular spacer and using the retrieval catheter to engage the coupling element.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE SEVERAL VIEWS OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-B illustrate the present device with the anchor portion collapsed (FIG. 1A) and expanded (FIG. 1B).

FIGS. 2A-C illustrate the anchor portion of the present device in an expanded state in a side view (FIG. 2A), isometric top view (FIG. 2B) and top view (FIG. 2C).

FIG. 3 illustrates the present device with the anchor portion engaged to tubular elements of the delivery system used for expanding the anchor.

FIGS. 4A-E illustrate delivery of the device, inflation of the spacer followed by expansion of the anchor.

FIG. 5 illustrates retrieval of the device using a lasso-like capture approach.

FIGS. 6A-C illustrate a prototype of the present device at various stages of anchor deployment.

FIG. 7 illustrates a heart model used for testing of the prototype device of FIGS. 6A-C.

FIGS. 8A-B illustrate in-silico testing results of the lobes (FIG. 8A) and flexible strut portions (FIG. 8B) of the present prototype.

FIG. 9 is a fluoroscopy image (short and long axis projections) taken during clinical investigation of a design prototype.

### DETAILED DESCRIPTION

The present invention is of a device which can be used to correct valvular insufficiency. Specifically, the present invention can be used to percutaneously treat valvular insufficiencies by delivering, positioning and anchoring a device that includes a trans-valvular spacer attached to a non-traumatic atrial anchor.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Implants and approaches for correcting valvular insufficiencies that result from incomplete leaflet coaptation have been described in the prior art. Such devices include atrial or ventricular anchors attached to spacers that are positioned within the valve opening to seal against the native valve leaflets when closed. Such implants are generally effective in sealing against the closed leaflets but are oftentimes delivered or anchored in a sub-optimal manner.

While reducing the present invention to practice, the present inventors have devised an implant and delivery system that address these limitations of prior art devices while providing numerous additional benefits in function and long-term stability. Specifically, the present delivery system and device enable a user to position and deploy the leaflet coaptation portion of the device (spacer) prior to deployment and anchoring of the anchor portion of the device thus optimizing coaptation prior to complete deployment of the device.

Thus, according to one aspect of the present invention there is provided a device (implant) for treating valvular insufficiency and delivery and retrieval catheter systems. As used herein, the phrase “valvular insufficiency” relates to any valve leaflet dysfunction that leads to valvular leakage and regurgitation. Valvular insufficiency can be caused by, for example, congenital heart disease, infection, leaflet stenosis, widening or stretching of the valve annulus, chordae rupture, dilated cardiomyopathy or valve prolapse.

The device of the present invention includes an expandable anchor designed for transitioning from a linear configuration (collapsed) to a torus-like configuration (expanded) and a transvalvular spacer that can be sized to achieve optimal leaflet coaptation in a regurgitant valve.

Spacer positioning and deployment (e.g., inflation to the correct diameter) is carried out prior to anchoring (i.e., without prior or co-deployment of the anchor portion) thus enabling a user to accurately position and inflate the spacer and verify optimal coaptation prior to unsheathing and anchoring of the anchor portion of the device.

The anchor can be fully or partially expanded using a dedicated mechanism positioned in the delivery catheter and thus can be released from the catheter outer tube without completely expanding and anchoring the device. Transitioning the anchor to an expanded state is carried out by maintaining position of a proximal end of the anchor while pushing the distal end using two tubular elements (push/pull rods) of the delivery system.

The device of the present invention further includes a transvalvular spacer attached to the distal end (opposite from delivery side) of the anchor. The spacer can be a solid spacer, a foam spacer, a fluid filled spacer or any other spacer that can provide a coaptation surface to valve leaflets (e.g., seal against the leaflets of a valve when closed). An embodiment of a spacer that includes a flexible column enclosed in an inflatable balloon is further described hereinbelow.

Delivery of the device of the present invention is carried out using a dedicated catheter system (delivery system) having several unique features. The delivery catheter includes two tubular elements for engaging two connectors on the anchor portion of the device. The two connectors are positioned opposite each other on distal and proximal ends of the anchor (at hubs 17 and 19 described below).

The present invention also includes a retrieval catheter and mechanism that enables retrieval of the device following deployment. Retrieval is carried out by lassoing or snaring a proximal end of the anchor using a dedicated catheter.

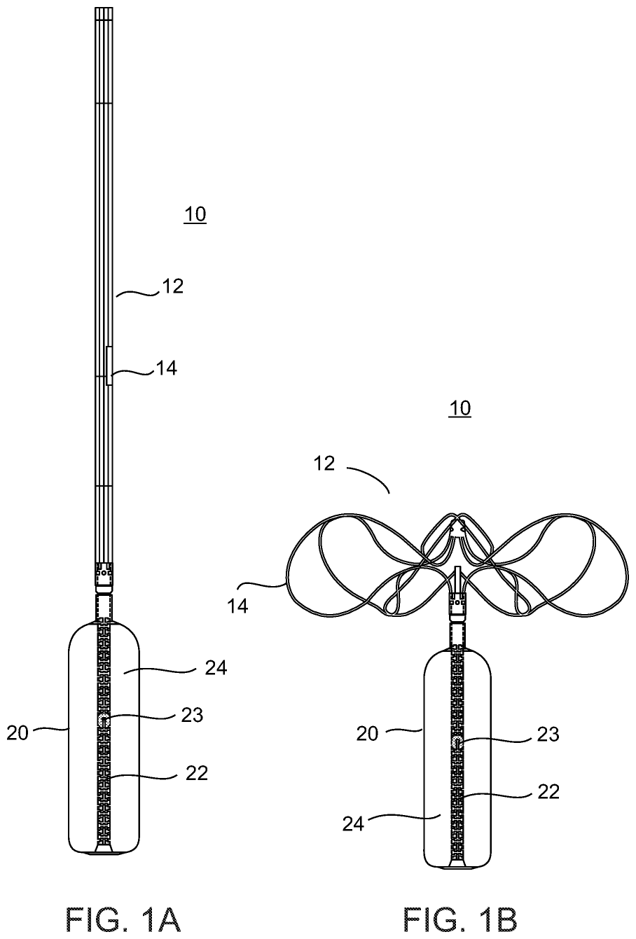

Referring now to the drawings, FIGS. 1-5 illustrate one embodiment of the present device which is referred to herein as device 10.

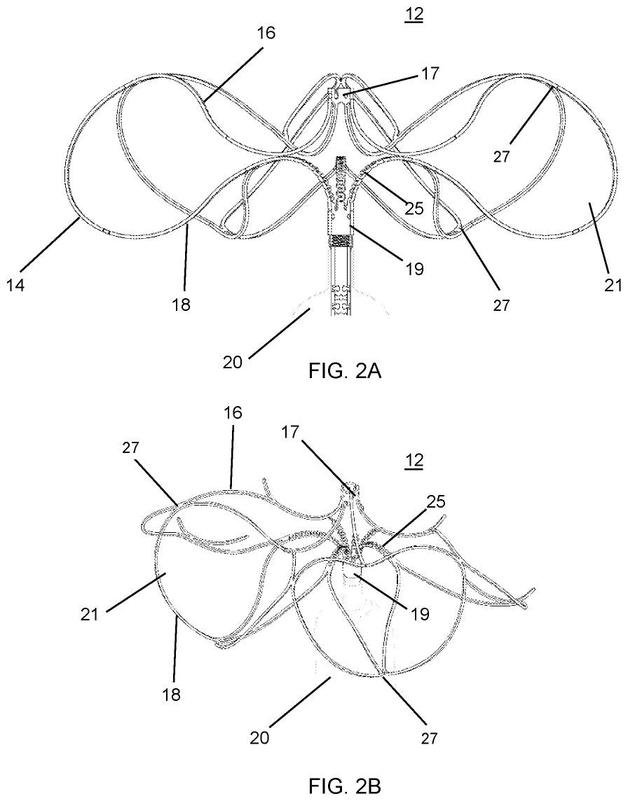

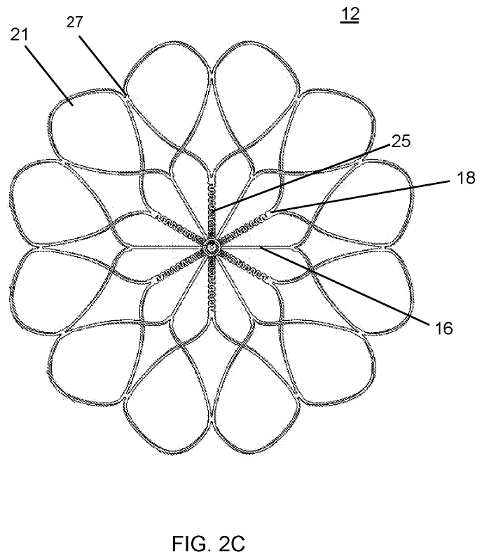

FIGS. 1A-B illustrate anchor 12 portion of device 10 in a collapsed (FIG. 1A) and expanded (FIG. 1B) states. FIGS. 2A-C illustrate three views of anchor 12.

Device 10 includes an anchor 12 attached to a spacer 20. Anchor 12 is constructed from struts 14 that are capable of transitioning from a linear configuration to a volume filling configuration. Struts 14 are fabricated from Nitinol or another alloy by, for example laser cutting a tube or sheet. Struts 14 are 0.3-0.6 in width and 0.3-0.6 mm in thickness. Anchor 12 is 50-90 mm in outer diameter (OD) and 13-45 mm in height when expanded and 3-5 mm in OD and 80-140 mm in length when collapsed.

As is shown in FIGS. 2A-C, struts 14 include six proximal struts 16 and six distal struts 18. Struts 16 branch out from a proximal central hub 17 while struts 18 branch out from a distal central hub 19. Struts 16 and 18 split to form lobes 21 that are interconnected at junctions 27. Although six struts are shown in FIGS. 2A-C, any number of struts 16, 18 (4, 5, 7, 8 or more each) can be implemented.

Anchor 12 can include 5-7 proximal and distal struts (6 shown) which when expanded form a toroidal space filling anchor. The function of the expanded shape is to apply a non-traumatic radial pressure on the chamber (e.g., atrium) inner walls in order to immobilize device 10. The anchor struts are designed to move from collapsed to expanded configuration without risk of tissue entanglement or snagging thanks to the ‘closed cell’ design-each lobe ends with a junction without open ends; thus, anchor increases in diameter in the heart chamber volume safely. The lobes are designed to be large with a large opening in order to avoid any hemodynamic disturbance and allow access to future devices through the anchor struts.

In order to enable spacer 20 to flex (tilt) with respect to anchor 12, struts 18 of anchor 12 include a flexible (e.g., undulating) portion 25 which is capable of elastically flexing thereby enabling spacer 20 to tilt (flex side to side) with respect to anchor 12.

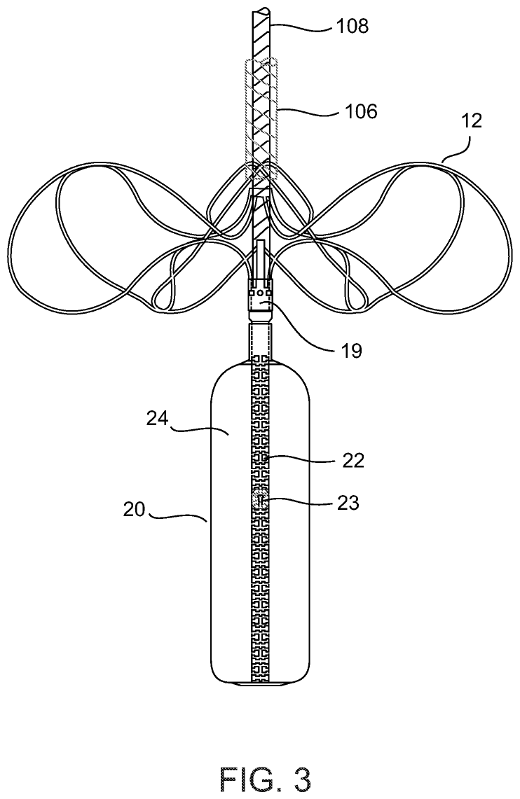

Spacer 20 shown in FIGS. 1A-B and FIG. 3 is constructed from a column 22 surrounded by an inflatable balloon 24.

Balloon 24 can be bonded to column 22 with ends turned outward or inverted inward. The latter configuration ensures that potentially traumatic implant edges do not face tissue.

Balloon 24 can be fabricated from a semi-compliant polymer using approaches well known in the art. Balloon 24 can be 30-55 mm in length and 10-20 mm in diameter (when fully inflated). The pressure range for balloon 24 can be from 0 atmospheres (atm) to 1 atm within normal operation.

The wall of balloon 24 can be non-permeable or semi-permeable (e.g., permeable to a fluid such as water but not cells). A permeable balloon wall can be used for osmotic filling as is described herein below.

Balloon 24 can be filled with a fluid (e.g., saline or an osmotic solution) through a single fluid port at the delivery catheter. Filling through the port can be carried out using a syringe attached to a proximal fluid port of the catheter. The balloon is filled using a saline contrast mixture at a ratio between 10-40% which maintains the balloon in hydrostatic and osmotic equilibrium with surrounding blood. Balloon 24 is filled through a valve 23 (FIG. 1A) covering the fluid port in the central column 22. Fluid is injected from a proximal syringe (positioned outside the body) through tube 108 of delivery system 100, into central column 22 and into balloon 20 through valve 23.

Balloon 24 can be filled to a final volume and pressure during delivery of device 10 (e.g., prior to expansion of anchor 12).

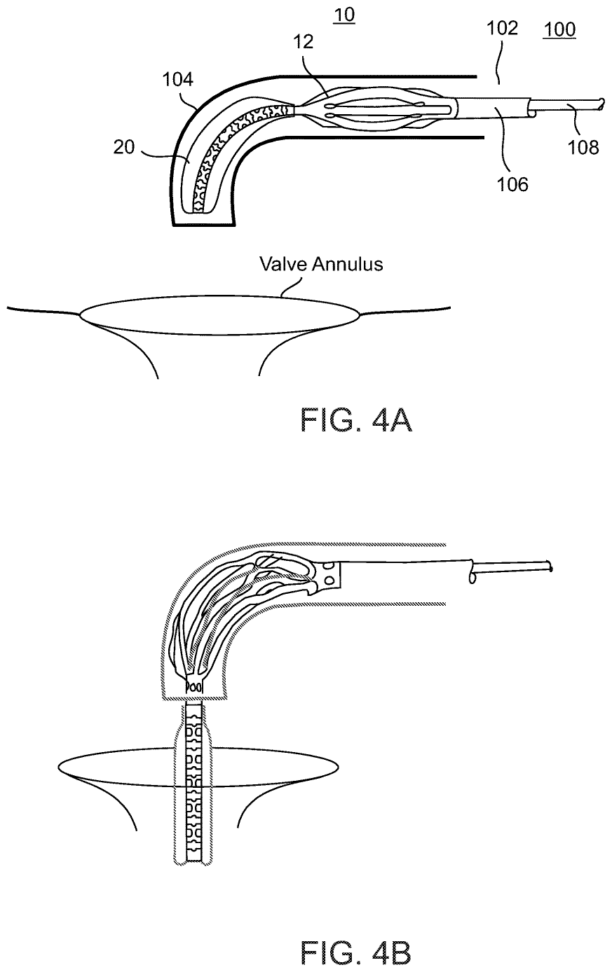

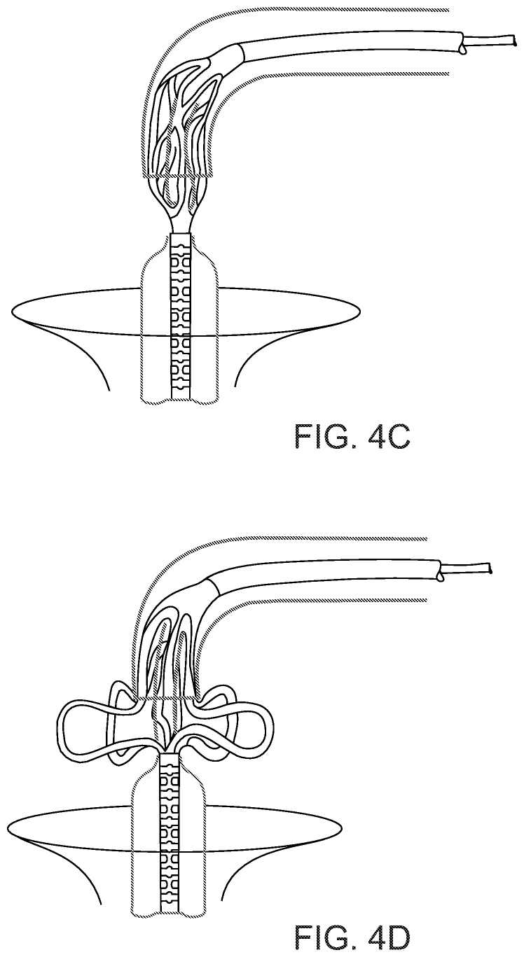

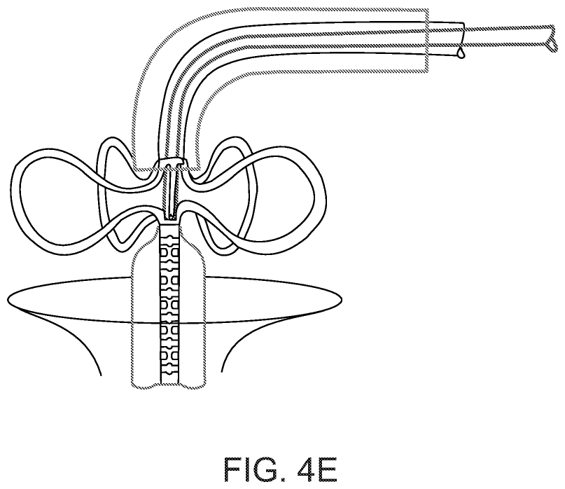

The efficacy of balloon 24 in leaflet coaptation can be tested prior to expansion of anchor 12. This can be achieved by deploying and expanding balloon 24 and testing for leaflet coaptation while device 10 is attached to the delivery system and anchor 12 is partially deployed or in a collapsed configuration. Such a procedure is described hereinbelow with reference to FIGS. 4A-E.

As is mentioned hereinabove, balloon 24 can be semi-permeable to allow for osmotic filling. Balloon 24 can be partially inflated with an osmotic solution (e.g., saline contrast solution) having an osmotic potential that is greater than that of blood (e.g., osmolarity of 300-500 milliosmoles/L).

The osmotic potential of blood is generally constant. Therefore, the osmotic potential of the solution used to fill balloon 24 will precisely determine the final hydrostatic pressure and volume of the balloon. The osmosis mechanism ensures that the fluid medium does not permeate out of balloon during use, thus maintain a constant long-term volume. In addition, the pressure that the balloon is filled to intraoperatively does not need to be accurate since the osmotic mechanism will induce water to permeate into balloon until reaching an equilibrium between hydrostatic and osmotic pressures.

Balloon 20 is connected to anchor 12 at hub 19. Column 22 is made of a nitinol tube with a laser-cut pattern designed to allow bending of the implant during transcatheter delivery but sufficiently stiff axially to support the compression load applied on balloon 20 during the cardiac cycle.

As is shown in FIG. 3, anchor 12 of device 10 is connected at hubs 17 and 19 to elements 106 and 108 (push/pull rods) of a delivery catheter system (further described hereinbelow with Reference to FIGS. 4A-E.

As is mentioned hereinabove, device 10 of the present invention is configured for correcting incomplete leaflet coaptation in a heart valve (valvular insufficiency). Device 10 can be used for correcting atrioventricular valve coaptation (bicuspid or tricuspid) by delivering device 10 using a catheter system.

Device 10 can be delivered via a percutaneous approach from the jugular vein through the superior vena cava or from the femoral vein through the inferior vena cava. In the case of the mitral/tricuspid valve, the preferred approaches would be femoral and transseptal.

FIGS. 4A-E illustrate delivery and deployment of device 10 using catheter system 100. Catheter system 100 is used to deliver device 10 as follows. Prior to delivery, system 100 is prepared on a benchtop sterile area by laying out system 100 components and accessories and testing each as well as the overall system. Device 10 is removed from packaging and loaded onto catheter system 100 by attaching device 10 to inner catheter 102 (includes elements 106 and 108) and sheathing device 10 and inner catheter 102 with sheath 104.

Catheter system 100 includes a mechanism for deploying anchor 12 that includes two translatable elements 106 and 108 (push/pull rods). The mechanism is actuatable via one or more levers/triggers positioned on a handle of catheter system 100. The mechanism can be used to fully deploy and optionally fully collapse anchor 12 and any expansion state in-between (partial deployment).

Such mechanical and controlled deployment is independent from positioning and deployment of spacer 20 and thus provides numerous benefits including:
(i) the ability to evaluate anchor 12 positioning in relation to atrium w/o trauma to tissue;
(ii) the ability to assess balloon 20 position in relation to anchor 12 and assess efficacy; and
(iii) the ability to maneuver and reposition anchor in atrium and even completely collapse and abort procedure, all prior to permanent implantation.

As is shown in FIG. 3, first element 106 is attachable to a proximal connector 13 of anchor 12 while second element 108 is attachable to a distal connector 15 of anchor 12. Translating elements 106 and 108 (within inner catheter 102) in opposite directions expands anchor 12.

Connectors 13 and 15 can be of nitinol or a biocompatible steel or a polymer. Connection between anchor 12 and delivery catheter elements 106 and 108 can be via flexible female grasper element radially compressed into locked position at hub 17 creating an axial attachment with some radial motion allowed. Another embodiment can include ‘male’ fingers normally in an expanded state. These can be radially compressed by delivery system into a locked position, to which they are threaded into ‘female’ slots and create an axially stiff connection to anchor 12. Another embodiment depicts sutures threaded through delivery system 100 and through hub 17. The sutures are tensioned to connect device 10 to delivery system 100, until deployment, following which the sutures are pulled away and device 10 is released from delivery system 100. Another embodiment can include a male thread at the edge of tube 106 and a female thread at hub 19 of anchor 12; attachment/detachment can be carried out by rotating element 106.

FIGS. 4A-E illustrate delivery of device 10 and sequential deployment of spacer 20 and anchor 12.

The procedure starts by inserting a standard guidewire through a vascular access site into target anatomy, for instance the right ventricle. An introducer sheath with dilator is introduced over the wire to the vessel creating an access port. The dilator is removed, and the delivery system and implant are loaded over the wire, through the introducer sheath to the treatment site. In the Tricuspid example to the right Atrium, or left Atrium in the Mitral side. As is shown in FIG. 4A, when delivery system 100 reaches target site, sheath 104 is used to position the implant above and perpendicular to valve annulus. Elements 106 and 108 (also referred to herein as tubes 106 and 108) are pushed and balloon is exposed, crossing the valve leaflets (FIG. 4B). Balloon 20 is inflated under fluoroscopy and sheath 104 is further employed to maneuver the balloon into desired position (FIG. 4C). Valve regurgitation is measured using echocardiography prior and post balloon inflation to assess efficacy; anchor 12 can then be deployed depending on the operator's preferences. Tube 106 and 108 are pushed simultaneity until sufficiently exposing anchor 12 (between 10-30 mm out of sheath), above the heart valve (FIG. 4D). Tube 108 is maintained in position [tension] distally while tube 106 is pushed under compression at the proximal end 17. This creates a total compression load on crown, pushes proximal connection 17 and causes an increase in diameter w/o translation of the distal end of anchor 19. Until this stage, the operator may choose to deflate balloon and completely re-sheath implant w/o trauma to patient. Alternately the surgeon may choose to detach connectors 15, 13 and permanently implant the device, at which the delivery system is removed and efficacy may be measured using echocardiogram.

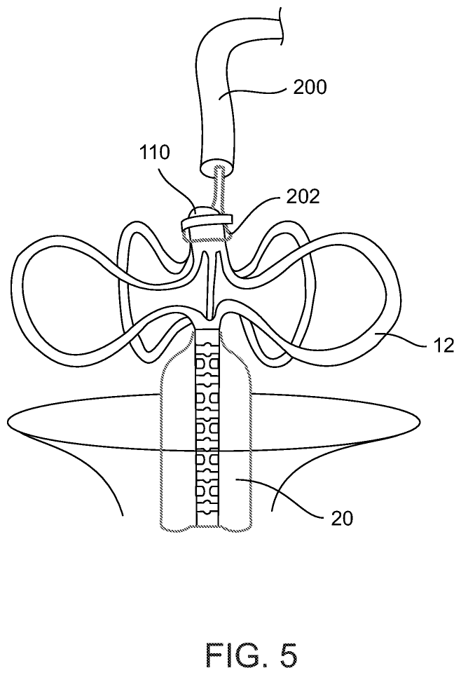

As is shown in FIG. 5, device 10 includes a coupling element 110 for engaging a retrieval catheter 200. Retrieval catheter 200 includes a lasso/snare 202. In the event operator wishes to remove implant immediately after implantation for safety or efficacy reasons, the retrieval catheter may be used as a bail out instead of open-heart surgery, to remove the implant after permanently detaching from delivery system. The retrieval catheter is introduced through any steerable catheter and under echo and fluoroscopic imaging captures the coupling element 110. As device 10 is gripped by lasso and pulled into catheter, a puncturing element deployed from catheter 200 can be used to puncture balloon 20 to deflate it.

As used herein the term “about” refers to ±10%.

Additional objects, advantages, and novel features of the present invention will become apparent to one ordinarily skilled in the art upon examination of the following examples, which are not intended to be limiting.

### EXAMPLES

Reference is now made to the following example, which together with the above descriptions, illustrate the invention in a non-limiting fashion.

### Prototype Testing

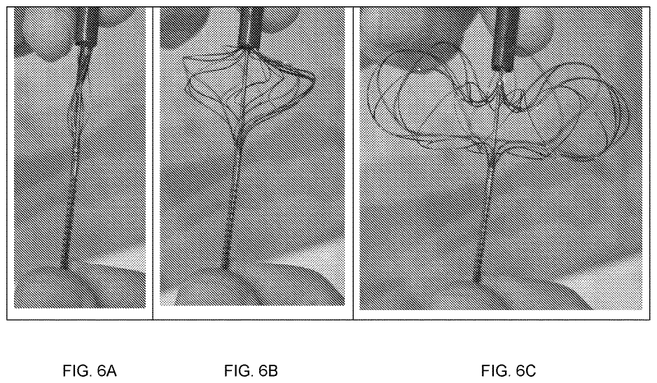

A prototype of the present device (FIGS. 6A-C) was manufactured using common approaches known in the art and demonstrated in a lab environment under physiological conditions that included implantation in an anatomically accurate flexible heart model (major blood vessels, atriums, ventricles, pulmonary arteries etc.) using a transcatheter approach.

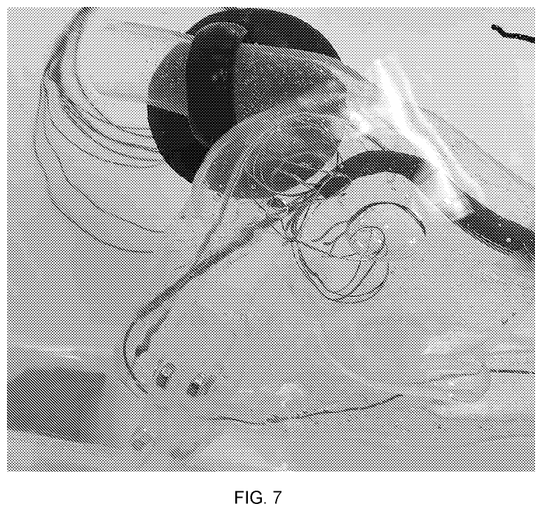

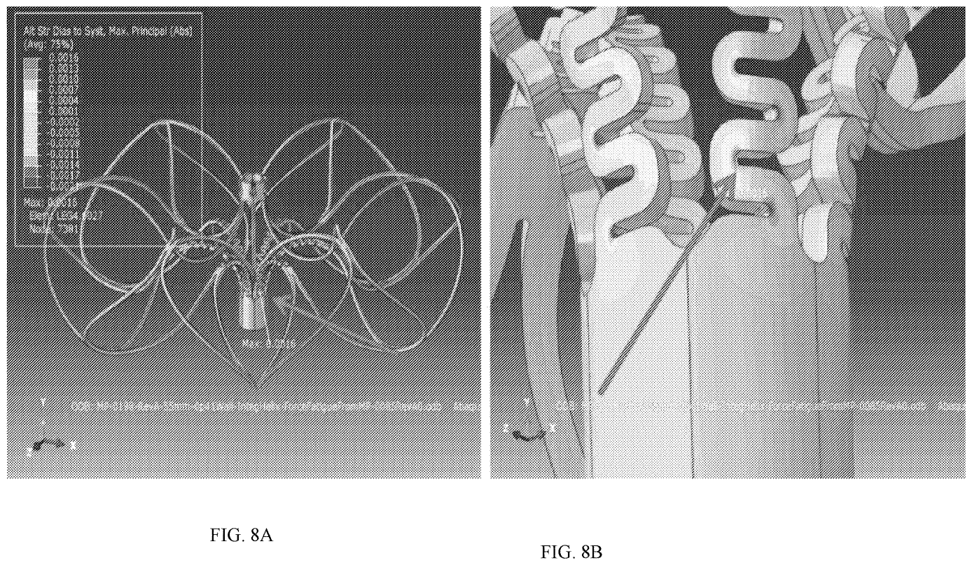

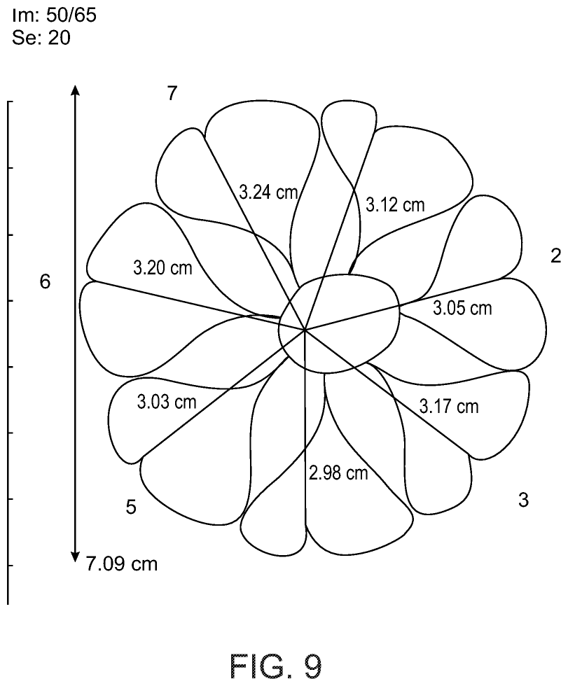

A heart model (FIG. 7) was manufactured from silicone/latex based on data from CT scans of heart failure patients typical of MR or TR. The model was placed in bath of body temperature water and the delivery system was preconditioned for 15 minutes. The test result demonstrates the system behaves as anticipated in this setting and was successfully deployed in the simulated target anatomy. Further testing has been conducted in-silico (FEA using ABACUS software) to show that the design is capable to withstand high cycle loading in the target anatomy (FIGS. 8A-B). Boundary conditions for in-silico testing were measured from fluoroscopy images (short and long axis projections) taken during clinical investigation of earlier generations of this design (FIG. 9). Furthermore, pre-clinical studies have demonstrated feasibility of implantation procedure in this setting.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A device for treating valvular insufficiency comprising:
(a) a transvalvular spacer for providing a coaptation surface to leaflets of a heart valve; and
(b) an expandable anchor attached to said transvalvular spacer and being for positioning said transvalvular spacer across said heart valve when expanded in a heart chamber, said expandable anchor having a proximal connector attachable to a first element of a delivery system and a distal connector attachable to a second element of a delivery system, said first and second elements being moveable in opposite directions to expand said expandable anchor.

2. The device of claim 1, wherein said transvalvular spacer maintains a position relative to said heart valve when said first element and said second element move in opposite directions.

3. The device of claim 1, wherein a degree of movement of said first element and said second element determines an extent of expansion of said anchor.

4. The device of claim 1, further comprising a coupling element for engaging a retrieval catheter.

5. The device of claim 4, wherein said coupling element is configured for engagement via a lasso.

6. A device for treating valvular insufficiency comprising:
(a) a transvalvular spacer for providing a coaptation surface to leaflets of a heart valve; and
(b) an expandable anchor attached to said transvalvular spacer and being for positioning said transvalvular spacer across said heart valve when expanded in a heart chamber, said expandable anchor including a plurality of struts, wherein some of said struts include a flexible portion for enabling said transvalvular spacer to tilt with respect to said expandable anchor when expanded.

7. The device of claim 6, wherein said flexible portion is an undulating portion.

8. A method of treating valvular insufficiency comprising:
(a) deploying within a heart valve a transvalvular spacer having a balloon for providing a coaptation surface to leaflets of said heart valve; and
(b) inflating said balloon within said heart valve to a volume that meets a coaptation goal;
(b) expanding an expandable anchor attached to said transvalvular spacer within a heart chamber to thereby anchor said transvalvular spacer across said heart valve.

9. The method of claim 8, wherein said transvalvular spacer maintains a position relative to said heart valve when said expandable anchor is expanded.

10. The method of claim 8, further comprising deflating and optionally reinflating said balloon following (b).

11. The method of claim 8, wherein said expandable anchor further comprises a coupling element for engaging a retrieval catheter.

12. The method of claim 11, further comprising deflating said transvalvular spacer following (b) and using said retrieval catheter to engage said coupling element.
